(19) United States
(12) Patent Application Publication
(10) Pub. No.: US 2019/0322734 A1
(43) Pub. Date: Oct. 24, 2019

(54) TREATMENT PARADIGM

(71) Applicant: GlaxoSmithKline Intellectual Property Development Limited, Brentford Middlesex (GB)

(72) Inventor: Paul-Peter TAK, Stevenage (GB)

(21) Appl. No.: 15/772,664

(22) PCT Filed: Oct. 31, 2016
(86) PCT No.: PCT/EP2016/076225

§ 371 Date: May 1, 2018

(30) Foreign Application Priority Data
Nov. 2, 2015 (GB) 1519331.1

(51) Int. Cl. C07K 16/24; A61K 39/395; A61K 31/519

(52) U.S. Cl. CPC C07K 16/243; A61K 39/3955; A61K 2039/545; C07K 2317/76; A61K 31/519

(57) ABSTRACT

An antibody antagonist of GM-CSF for use in the treatment of a patient suffering from rheumatoid arthritis (RA), wherein said antibody is administered to said patient according to the following treatment regimen:
i. a first period wherein the antibody is administered once a week; and
ii. a second period wherein the antibody is administered every other week and then ceased once said patient has sustained remission for a continuous period of at least two months.

### FIELD OF THE INVENTION

The present invention provides antibody antagonists of GM-CSF for use in the treatment of rheumatoid arthritis (RA), in particular early RA, and methods for the treatment of RA, in particular early RA using such antibodies. Antibody antagonists of GM-CSF, in particular MOR103, namilumab and mavrilimumab, are administered to patients suffering from RA, in particular early RA according to a specific treatment paradigm to achieve remission, while limiting the period the patient receives treatment with said antibody.

### BACKGROUND TO THE INVENTION

RA is a chronic systemic inflammatory disease that affects more than twenty million people world wide, up to 1% of the adult population (Gabriel et al.; 2001). RA primarily affects the joints and is characterized by chronic inflammation of the synovial tissue, which eventually leads to the destruction of cartilage, bone and ligaments and can cause joint deformity. RA has a peak incidence between 40 and 60 years of age and affects primarily women. The cause of RA is not known, however, certain histocompatibility antigens are associated with poorer outcomes.

The management of rheumatoid arthritis (RA) rests primarily on the use of disease-modifying antirheumatic drugs (DMARDs). These agents are the cornerstone of RA treatment throughout all stages of the disease and are commonly characterised by their capacity to reduce or reverse signs and symptoms, disability, impairment of quality of life, inability to work, and progression of joint damage and thus to interfere with the entire disease process. DMARDs form two major classes: synthetic chemical compounds (csDMARDs) and biological agents (bDMARDs).

Current recommendations for management of RA with synthetic and biological disease-modifying anti-rheumatic drugs (csDMARDS and bDMARDS respectively) have been published by the European League Against Rheumatism (EULAR) (Smolen J. S. et al.; 2014).

It is recommended that therapy with DMARDs should be started as soon as a diagnosis of RA is made and they include starting treatment with conventional synthetic disease modifying anti-rheumatic drugs (csDMARDs). Methotrexate is the most widely used csDMARD and is a highly effective agent both as monotherapy and in combination with glucocorticoids, but other agents include hydroxychloroquine, sulfasalazine, gold salts, minocycline and leflunomide. Low dose glucocorticoids should be considered as part of the initial treatment strategy in combination with one or more csDMARDs for up to 6 months but it is recommended they are taperrd as soon as clinically feasible. NSAIDs may be recommended to be prescribed in combination with a csDMARD at low doses, to avoid adverse events, but they only provide symptomatic relief.

Where the patient does not achieve an improvement within six months, it is standard practice for the therapy to be adapted or changed. Such an adaption or change would usually be to replace the csDMARD with another csDMARD or add a further csDMARD in combination, both with addition of a low dose NSAID or glucocorticoid. Another course of treatment that may be considered, in particular where prognostically unfavourable factors are present, for example, very high disease activity or early joint damage, the EULAR recommendations suggest the addition of a biologic agent to the csDMARD.

Biologic agents for the treatment of RA include antibodies that target the following: tumour necrosis factor alpha (TNF-α), for example adalimumab, etanercept and infliximab; B-cells, for example rituximab (anti-CD20); T-cells, for example abatacept; and IL-6R, for example tocilizumab. If there is no improvement within six months, the EULAR recommendations advise replacement of the biologic agent with a second biologic agent or the addition of tofacitinib, a janus kinase (JAK) inhibitor, where two biologics have failed.

Questions have been raised in regards to the safety of biologic agents. Patients treated with some biologic agents have an increased risk of serious bacterial infection compared to patients treated with non-biologic agents. Blockade of the TNF-α pathway has been associated with an increased risk of infection, in particular tuberculosis reactivation (Scheinfeld N. et al.; 2004). Furthermore, many patients do not respond to current biologics or the therapeutic benefit is lost over time. In a study with a combination of methotrexate and etanercept (an anti-TNF-α biologic) only half the patients treated with the combination successfully achieved clinical remission as judged by DAS28 (Emery P. et al.; 2008).

The current “step-up” treatment paradigm of the addition of a biologic to a csDMARD after treatment with a csDMARD or combination of csDMARDS (optionally including treatment with a glucocorticoid or NSAID), is far from optimal with a substantial number of patients failing to respondor have an inadequate response and existing therapies have not been successful in getting sufficient numbers of patients into remission. Therefore new, safer and more effective therapies are required, particularly those directed at inducing a sustained remission that can be maintained on conventional DMARDs alone The present invention addresses this need.

### SUMMARY OF THE INVENTION

Many cells types (e.g. fibroblasts, macrophages, T and B lymphocytes and neutrophils) and mediators (e.g. cytokines) have been implicated in RA. A key role for macrophages has been suggested in part by successful treatment of RA in some patients with the blockade of TNF-α, which is widely considered to be produced by activated macrophages in inflamed tissue (Kinne R. W. et al., 2007). It has been observed that the number of macrophages in the synovial tissue correlates with the degree of joint erosion (Mulherin D. et al., 1996) and that increased numbers of macrophages are an early hallmark of active disease (Tak P. P. et al., 2000). It has also been found that the depletion of macrophages from inflamed tissue and the circulation can have profound benefit on patients (Barrer P. et al., 2000; and Kashiwagi, N. et al., 2002). Colony-stimulating factors (CSFs) have been suggested for a potential point of intervention for inflammatory disorders, such as RA (reviewed e.g. in Hamilton J. A., 2008; and Cornish A. L. et al.; 2009). One such CSF is granulocyte-macrophage colony-stimulation factor (GM-CSF).

GM-CSF is a known driver in RA and is a key regulator of macrophages and their precursors in bone marrow, peripheral blood and synovial tissue. GM-CSF is involved in controlling the mobilisation and trafficking of macrophages from the circulation into joints; once in the joints GM-CSF controls activation of immature macrophages and drives maturation. This is illustrated in FIG. 1.

GM-CSF induces the proliferation and activation of macrophage lineage cells leading to strongly increased production of key proinflammatory cytokines (including TNF-α, IL-6, and IL-1), chemokines and matrix degrading proteases (Fleetwood et al., 2007; Gasson et al., 1991; Hamilton et al., 2004; Hamilton et al., 2013; Hart et al., 1991; Mantovani et al., 2007). By targeting GM-CSF early on in disease progression, preferably within 2 years of onset of symptoms, the number of macrophages entering the synovium, proliferating and surviving would be minimised. This reduction would significantly reduce inflammatory joint damage and subsequent functional joint impairment thus achieving higher levels of remission than current therapies. Once such damage occurs a self-sustaining cycle of inflammation begins which is more difficult to treat due to the large number of mediators and mechanisms of action involved.

In later stage RA it has been suggested that p53 tumour suppressor gene mutations and other key regulator genes could help convert chronic synovitis into an autonomous disease, independent of the initial immune-mediated inflammatory process. Furthermore the cumulative destruction of bone and articular cartilage may result in the release of fragments that enhance inflammation (Tak, P. P., 2001). It is therefore important to treat a patient with an antagonist for GM-CSF early in disease progression, during the ‘therapeutic window of opportunity’ before increased synovial tissue mass, progressive joint destriction and any epigenetic changes, thereby increasing the likelihood of achieving remission. Initiation of treatment with an antagonist for GM-CSF, preferably in combination with one or more csDMARDs and optionally glucocorticoids and/or NSAIDs, as opposed to the convention treatment paradigm of one or more csDMARDs and optionally glucocorticoids and/or NSAIDs followed by later add-on treatment with a biologic, would be a more effective treatment for patients with RA, especially with early RA.

The present invention provides for the first time a treatment paradigm which more readily addresses the benefit-risk balance by providing an on-biologic remission induction phase and subsequent off-biologic remission maintenance phase treatment paradigm, with a reduced exposure to biologic treatment over an individual patient's lifetime, translating into a better safety profile with reduced long-term risks of infection and malignancy, the overall burden of treatment, as well as the costs of therapy. The new treatment paradigm is capable of switching the course of the RA disease to a more benign form where remission is maintained without the need for long-term biologic therapy.

Achieving remission is important as it provides relief from the signs and symptoms of RA, (pain, swelling, stiffness and fatigue), prevents the progression of joint damage and restores functional capacity; and prevents long term morbidity and mortality, for example due to cardiovascular complications, malignancy and infection.

In one aspect, the invention provides an antibody antagonist of GM-CSF for use in the treatment of a patient suffering from RA, wherein said antibody is administered to said patient according to the following treatment regimen:
i. a first period wherein the antibody is administered once a week; and
ii. a second period wherein the antibody is administered every other week and then ceased once said patient has sustained remission for a continuous period of at least two months.

In another aspect the invention provides the use of an antibody antagonist of GM-CSF in the manufacture of a medicament for use in the treatment of a patient suffering from RA, wherein said antibody is administered to said patient according to the following treatment regimen:
iii. a first period wherein the antibody is administered once a week; and
iv. a second period wherein the antibody is administered every other week and then ceased once said patient has sustained remission for a continuous period of at least two months.

In another aspect, the invention provides a method for the treatment of RA in a subject comprising administration to the subject an effective amount of an antibody antagonist of GM-CSF, wherein said antibody is administered to said patient according to the following treatment regimen:
i. a first period wherein the antibody is administered once a week; and
ii. a second period wherein the antibody is administered every other week and then ceased once said patient has sustained remission for a continuous period of at least two months.

In one embodiment the patient is a human patient.

In one embodiment remission is maintained after the second period for at least six months while treatment with the antibody is ceased. In another embodiment remission is maintained after the second period for at least one year while treatment with the antibody is ceased.

In one embodiment the first period is at least 4 weeks. In one embodiment the first period is 4, 5, 6, 7, 8, 9 or 10 weeks. In one embodiment the first period is five weeks.

In one embodiment the first period is five weeks and the antibody is administered on days 1, 8, 15, 22 and 29 of the first period.

In one embodiment the second period is from one to two years

In one embodiment, the second period starts directly after the end of the first period (e.g. if the first period is 5 weeks long, the second period begins on day 1 of week 6). In a further embodiment the second period starts one week after the end of the first period (e.g. if the first period is 5 weeks long, the second period begins on day 1 of week 7).

In one embodiment the first period is five weeks and the antibody is administered on days 1, 8, 15, 22 and 29 of the first period and the second period is from one to two years, the second period beginning with dosing after the end of week 6 on day 43, (day 1 of week 7) as measured from the first day of the first period.

In another embodiment the antibody must be administered on the same day each week ±1 day for the first period. For the second period, the antibody must be administered on the same day every other week ±3 days.

In one embodiment the antibody treatment in the second period is ceased once the patient has sustained remission for a continuous period of from two months to one year. In one embodiment the antibody treatment in the second period is ceased once the patient has sustained remission for a continuous period of at least two months, for example two months. In another embodiment the antibody treatment in the second period is ceased once the patient has sustained remission for a continuous period of at least three months, for example three months. In another embodiment the antibody treatment in the second period is ceased once the patient has sustained remission for a continuous period of at least four months, for example four months. In another embodiment the antibody treatment in the second period is ceased once the patient has sustained remission for a continuous period of at least five months, for example five months. In another embodiment the antibody treatment in the second period is ceased once the patient has sustained remission for a continuous period of at least six months, for example six months. In another embodiment the antibody treatment in the second period is ceased once the patient has sustained remission for a continuous period of at least one year, for example one year. In another embodiment the antibody treatment in the second period is ceased once the patient has sustained remission for a continuous period of at least 13 months, for example 13 months. In another embodiment the antibody treatment in the second period is ceased once the patient has sustained remission for a continuous period of at least 14 months, for example 14 months. In another embodiment the antibody treatment in the second period is ceased once the patient has sustained remission for a continuous period of at least 15 months, for example 15 months. In another embodiment the antibody treatment in the second period is ceased once the patient has sustained remission for a continuous period of at least 16 months, for example 16 months. In another embodiment the antibody treatment in the second period is ceased once the patient has sustained remission for a continuous period of at least 17 months, for example 17 months. In another embodiment the antibody treatment in the second period is ceased once the patient has sustained remission for a continuous period of at least 18 months, for example 18 months. In another embodiment the antibody treatment in the second period is ceased once the patient has sustained remission for a continuous period of at least 19 months, for example 19 months. In another embodiment the antibody treatment in the second period is ceased once the patient has sustained remission for a continuous period of at least 20 months, for example 20 months. In another embodiment the antibody treatment in the second period is ceased once the patient has sustained remission for a continuous period of at least 21 months, for example 21 months. In another embodiment the antibody treatment in the second period is ceased once the patient has sustained remission for a continuous period of at least 22 months, for example 22 months. In another embodiment the antibody treatment in the second period is ceased once the patient has sustained remission for a continuous period of at least 23 months, for example 23 months. In another embodiment the antibody treatment in the second period is ceased once the patient has sustained remission for a continuous period of at least 2 years, for example 2 years.

In one embodiment the median plasma concentration of the antibody is maintained above 3 μg/mL during the first period.

In one embodiment the median plasma concentration of the antibody is maintained above 3 μg/mL during the second period.

In one embodiment the maximum plasma concentration reached during the first period is at least 7 μg/mL.

In one embodiment the maximum plasma concentration reached during the second period is at least 5 μg/mL.

This is to say that, in each period, at some time throughout the period the maximum plasma concentration is reached. This is demonstrated in FIG. 2 is the predicted pharmacokinetic plasma (PK) profile for MOR103 according to a dosage regimen of five fixed loading doses of 180 mg, subcutaneously, administered every week on days 1, 8, 15, 22 and 29, followed by maintenance fixed doses of 180 mg subcutaneously administered every other week on days 43, 57 and 71 (Week 10).

In one embodiment RA is early RA.

In one embodiment the patient is csDMARD-naïve before commencing treatment.

In one embodiment the patient receives csDMARD treatment in combination with the antibody treatment which is continued after the second period. In one embodiment the csDMARD is administered to said patient once a week. In one embodiment the csDMARD is methotrexate.

### DESCRIPTION OF DRAWINGS/FIGURES

FIG. 1 depicts the role of GM-CSF in RA pathogenesis and summarizes why GM-CSF is a prime target, especially in early disease.

FIG. 2 is the predicted pharmacokinetic plasma (PK) profile for MOR103 according to a dosage regimen of five fixed loading doses of 180 mg, subcutaneously, administered every week on days 1, 8, 15, 22 and 29, followed by maintenance fixed doses of 180 mg subcutaneously administered every other week on days 43, 57 and 71 (Week 10).

FIG. 3 is simulated MOR103 serum concentration-time profiles with 5 weekly doses followed by every other week dosing

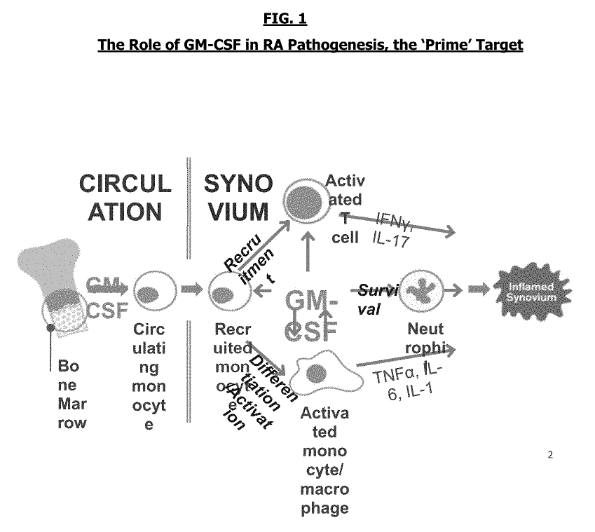

### DETAILED DESCRIPTION OF THE INVENTION

### Definitions

The term “antibody” is used in the broadest sense and specifically covers monoclonal antibodies, polyclonal antibodies, multispecific antibodies (e.g. bispecific antibodies) formed from at least two intact antibodies, and antibody fragments so long as they exhibit the desired biological activity. Such an antibody may be chimeric, humanized or a human antibody. In one embodiment the antibody is chimeric. In another embodiment the antibody is humanized. In a further embodiment the antibody is human.

“Antibody fragments” herein comprise a portion of an intact antibody which retains the ability to bind antigen. Examples of antibody fragments include Fab, Fab′, F(ab′)2, and Fv fragments; diabodies; linear antibodies; single-chain antibody molecules; and multispecific antibodies formed from antibody fragments.

The term “monoclonal antibody” as used herein refers to an antibody obtained from a population of substantially homogeneous antibodies, i.e., the individual antibodies comprising the population are identical and/or bind the same epitope, except for possible variants that may arise during production of the monoclonal antibody, such variants generally being present in minor amounts. In contrast to polyclonal antibody preparations that typically include different antibodies directed against different determinants (epitopes), each monoclonal antibody is directed against a single determinant on the antigen. In addition to their specificity, the monoclonal antibodies are advantageous in that they are uncontaminated by other immunoglobulins. The monoclonal antibodies herein specifically include chimeric” antibodies (immunoglobulins) in which a portion of the heavy and/or light chain is identical with or homologous to corresponding sequences in antibodies derived from a particular species or belonging to a particular antibody class or subclass, while the remainder of the chain(s) is identical with or homologous to corresponding sequences in antibodies derived from another species or belonging to another antibody class or subclass, as well as fragments of such antibodies, so long as they exhibit the desired biological activity.

“Humanized” forms of non-human (e.g., murine) antibodies are chimeric antibodies that contain minimal sequence derived from non-human immunoglobulin. For the most part, humanized antibodies are human immunoglobulins (recipient antibody) in which residues from a hypervariable region of the recipient are replaced by residues from a hypervariable region of a non-human species (donor antibody) such as mouse, rat, rabbit or nonhuman primate having the desired specificity, affinity, and capacity. In some instances, framework region (FR) residues of the human immunoglobulin are replaced by corresponding non human residues. Furthermore, humanized antibodies may comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable regions correspond to those of a non-human immunoglobulin and all or substantially all of the FRs are those of a human immunoglobulin sequence, except for FR substitution(s) as noted above. The humanized antibody optionally also will comprise at least a portion of an immunoglobulin constant region, typically that of a human immunoglobulin.

A “human antibody” herein is one comprising an amino acid sequence structure that corresponds with the amino acid sequence structure of an antibody obtainable from a human B-cell, and includes antigen-binding fragments of human antibodies. Such antibodies can be identified or made by a variety of techniques, including, but not limited to: production by transgenic animals (e.g., mice) that are capable, upon immunization, of producing human antibodies in the absence of endogenous immunoglobulin; selection from phage display libraries expressing human antibodies or human antibody; generation via in vitro activated B; and isolation from human antibody producing hybridomas.

An antibody “antagonist of GM-CSF” is an antibody that inhibits the activity or function of GM-CSF (Granulocyte-macrophage colony-stimulating factor). The term includes antibodies specifically binding to GM-CSF and antibodies that specifically bind to the GM-CSF receptor.

The term antibody “specific for GM-CSF” or “anti-GM-CSF antibody” refers to an antibody which binds to GM-CSF; and inhibits the activity or function of GM-CSF.

The term antibody “specific for the GM-CSF receptor” refers to an antibody which binds to the GM-CSF receptor, for example the α-chain of the GM-CSF receptor; and inhibits the activity or function of GM-CSF. Preferably the binding affinity for antigen is of Kd value of 10′ mol/l or lower (e.g. 10′″ mol/l), preferably with a Kd value of 10′″ mol/l or lower (e.g. 10′″ mol/l). The binding affinity is determined with a standard binding assay, such as surface plasmon resonance technique (BIACORE).

A patient who is “csDMARD-naïve” is one who has never been administered a csDMARD.

The “DAS28” is the disease activity score of twenty-eight joints and is used to monitor disease progression. The joints included in DAS28 are (bilaterally): proximal interphalangeal joints (ten joints), metacarpophalangeal joints (ten joints), wrists (two joints), elbows (two joints), shoulders (two joints) and knees (two joints). When looking at these joints, both the number of joints with tenderness upon touching (TEN28) and swelling (SW28) are counted. In addition, the erythrocyte sedimentation rate (ESR) and/or the C-Reactive Protein (CRP) value is measured. Also, the affected person makes a subjective assessment (SA) of disease activity during the preceding 7 days on a scale between 0 and 100, where 0 is “no activity” and 100 is “highest activity possible”. With these parameters, DAS28 is calculated as:

DAS28(CRP)=0.56×√(TEN28)+0.28×√(SW28)+0.014×SA+0.36×ln(CRP+1)+0.96;

DAS28(ESR)=0.56×√(TEN28)+0.28×√(SW28)+0.014×SA+0.70×ln(ESR).

As used herein, the term “early rheumatoid arthritis” or “early RA” is a disease duration of years from onset of symptoms and/or diagnosis

The “EULAR response criteria” is a comparison of the DAS28 from one patient on two different time points, to define improvement or response. The EULAR response criteria are defined as follows:

| | DAS28 improvement | | |
|---|---|---|---|
| Present DAS28 | >1.2 | >0.6 and ≤1.2 | ≤0.6 |
| ≤3.2 | good response | moderate response | no response |
| >3.2 and ≤5.1 | moderate response | moderate response | no response |
| >5.1 | moderate response | no response | no response |

A “loading period” is when an initial higher dose of the antibody is given at the beginning of the course of treatment to ensure the antibody reaches a therapeutic level.

The term “on-biologic remission induction phase” is the period where a patient is administered a fixed dose of an antibody antagonist of GM-CSF to bring about remission.

The term “off-biologic remission maintenance phase” is the period where the patient is not administered an antibody antagonist of GM-CSF or indeed any other antibody, but remission is continued.

The term “remission” as used herein is a disease activity score (DAS28), ((ESR) or (CRP)) of less than 2.6.

The term “sustained remission” as used herein means the presence of DAS28 scores less than 2.6 consistently for at least two months in consecutive measurements, at baseline and then monthly (Martire M. V. et al.; 2015).

### Antibody Antagonists of GM-CSF

Antibody antagonists of GM-CSF used in the methods and compositions of the invention include any antibody that inhibits the activity or function of GM-CSF In certain embodiments, the antibody used in the present invention is a monoclonal antibody. In other embodiments, the antibody used in the present invention is a chimeric, a humanized or a human antibody. In preferred embodiments, the antibody used in the present invention is a human antibody.

Suitable antibodies include for example MOR103, namilumab and mavrilimumab.

MOR103 is a fully human anti-GM-CSF antibody (Mol. Immunol. (2008) 46, 135-44; WO 2006/122797, WO2014/044768). Other synonyms for this antibody are MOR4357 and MOR04357. MOR103 is in a clinical Phase IIb trial for RA.

Namilumab is another fully human anti-GM-CSF antibody (WHO Drug Information, Vol. 24, No. 4, 2010, pages 382-383; WO 2006/111353 A1). Namilumab is being developed by Takeda/Amgen and is currently in Phase II for the treatment of RA and psoriasis.

Mavrilimumab (formerly CAM-3001) is a human monoclonal antibody that targets the alpha chain of the GM-CSF receptor (WHO Drug Information, Vol. 23, No. 4, 2009 pages 335-336; WO 2007/110631A1). Mavrilimumab completed Phase IIb studies in 2014 and is being developed by MedImmune (AstraZeneca).

In one embodiment the antibody is specific for GM-CSF. In other embodiments, the antibody used in the present invention is an antibody specific for a polypeptide encoding an amino acid sequence comprising SEQ ID NO.: 15.

In one embodiment the antibody specific for GM-CSF is an antibody comprising the heavy and light chain CRD's of MOR103 as defined by any method (e.g. Kabat et al. 1983 or Chothia et al. 1987) In one embodiment the sequences are defined by the Kabat method and are

CDRH1:
SEQ IN NO: 16
SYWMN
CDRH2:
SEQ IN NO: 17
GIENKYAGGATYYAASVKG
CDRH3:
SEQ IN NO: 18
GFGTDF
CDRL1:
SEQ IN NO: 19
SGDSIGKKYAY
CDRL2:
SEQ IN NO: 20
KKRPS
CDRL3:
SEQ IN NO: 21
SAWGDKGMV

In one embodiment the antibody specific for GM-CSF is an antibody comprising an HCDR1 region of sequence GFTFSSYWMN (SEQ ID NO.: 1), an HCDR2 region of sequence GIENKYAGGATYYAASVKG (SEQ ID NO.: 2), an HCDR3 region of sequence GFGTDF (SEQ ID NO.: 3), an LCDR1 region of sequence SGDSIGKKYAY (SEQ ID NO.: 4), an LCDR2 region of sequence KKRPS (SEQ ID NO.: 5), and an LCDR3 region of sequence SAWGDKGM (SEQ ID NO.: 6). In another embodiment the antibody comprises a heavy chain variable region peptide sequence according to SEQ ID NO.: 7 and a light chain variable regionpeptide sequence according to SEQ ID NO.: 8. In a further embodiment the antibody specific for GM-CSF is MOR103, having the heavy and light chain sequences in SEQ ID NO:14 and 15.

In other embodiments, the antibody used in the present invention is an antibody which cross competes with an antibody comprising an HCDR1 region of sequence GFTFSSYWMN (SEQ ID NO.: 1), an HCDR2 region of sequence GIENKYAGGATYYAASVKG (SEQ ID NO. 2), an HCDR3 region of sequence GFGTDF (SEQ ID NO.: 3), an LCDR1 region of sequence SGDSIGKKYAY (SEQ ID NO.: 4), an LCDR2 region of sequence KKRPS (SEQ ID NO.: 5), and an LCDR3 region of sequence SAWGDKGM (SEQ ID NO.: 6). In other embodiments, the antibody used in the present invention is an antibody which binds to the same epitope like an antibody specific for GM-CSF comprising an HCDR1 region of sequence GFTFSSYWMN (SEQ ID NO.: 1), an HCDR2 region of sequence GIENKYAGGATYYAASVKG (SEQ ID NO.: 2), an HCDR3 region of sequence GFGTDF (SEQ ID NO.: 3), an LCDR1 region of sequence SGDSIGKKYAY (SEQ ID NO.: 4), an LCDR2 region of sequence KKRPS (SEQ ID NO.: 5), and an LCDR3 region of sequence SAWGDKGM (SEQ ID NO.: 6).

In another embodiment the antibody specific for GM-CSF is an antibody comprising a heavy chain peptide sequence according to SEQ ID NO.: 11 and a light chain peptide sequence according to SEQ ID NO.: 12. In a further embodiment the antibody specific for GM-CSF is namilumab. In other embodiments, the antibody used in the present invention is an antibody which cross competes with an antibody comprising a heavy chain peptide sequence according to SEQ ID NO.: 11 and a light chain peptide sequence according to SEQ ID NO.: 12. In other embodiments, the antibody used in the present invention is an antibody which binds to the same epitope as an antibody comprising a heavy chain peptide sequence according to SEQ ID NO.: 11 and a light chain peptide sequence according to SEQ ID NO.: 12

In one embodiment the antibody is specific for the GM-CSF receptor. In one embodiment the antibody specific for the GM-CSF receptor is an antibody comprising a variable heavy chain peptide sequence according to SEQ ID NO.: 9 and a variable light chain peptide sequence according to SEQ ID NO.: 10. In a further embodiment the antibody specific for the GM-CSF receptor is mavrilimumab. In other embodiments, the antibody used in the present invention is an antibody which cross competes with an antibody comprising a heavy chain peptide sequence according to SEQ ID NO.: 9 and a light chain peptide sequence according to SEQ ID NO.: 10. In other embodiments, the antibody used in the present invention is an antibody which binds to the same epitope as an antibody comprising a heavy chain peptide sequence according to SEQ ID NO.: 9 and a light chain peptide sequence according to SEQ ID NO.: 10

### Pharmaceutical Compositions/Routes of Administration/Dosages

Therapeutic formulations of the antibodies of the present invention are prepared for storage by mixing the antibody having the desired degree of purity with optional pharmaceutically acceptable carriers, excipients or stabilizers in the form of lyophilized formulations or aqueous solutions. Acceptable carriers, excipients, or stabilizers are nontoxic to recipients at the dosages and concentrations employed, and include buffers such as phosphate, citrate, histidine and other organic acids; antioxidants including ascorbic acid and methionine; preservatives such as octadecyldimethylbenzyl ammonium chloride; hexamethonium chloride; benzalkonium chloride, benzethonium chloride; phenol, butyl or benzyl alcohol; alkyl parabens such as methyl or propyl paraben; catechol; resorcinol; cyclohexanol; 3-pentanol; and m-cresol; low molecular weight (less than about 10 residues) polypeptides; proteins, such as serum albumin, gelatin, or immunoglobulins; hydrophilic polymers such as polyvinylpyrrolidone; amino acids such as glycine, glutamine, asparagine, histidine, arginine, or lysine; monosaccharides, disaccharides, and other carbohydrates including glucose, mannose, or dextrins; chelating agents such as EDTA; sugars such as sucrose, mannitol, trehalose or sorbitol; salt-forming counter-ions such as sodium; metal complexes such as Zn-protein complexes; and/or non-ionic surfactants such as TWEEN™ (for example, Tween-80), PLURONICS™ or polyethylene glycol (PEG).

In one embodiment, the present invention provides a pharmaceutical composition comprising an antibody antagonist of GM-CSF and one or more pharmaceutically acceptable carriers and/or excipients for use in the treatment of a patient suffering from RA, wherein said pharmaceutical composition is administered to said patient according to the following treatment regimen:
i. a first period wherein the antibody is administered once a week; and
ii. a second period wherein the antibody is administered every other week and then ceased once said patient has sustained remission for a continuous period of at least two months.

In one embodiment the pharmaceutical composition comprising an antibody antagonist of GM-CSF and a pharmaceutically acceptable carrier and/or excipient comprises histidine, sorbitol and Tween-80.

The antibodies of the invention can be administered by any suitable means, such possible routes of administration include intramuscular, intravenous, intrarterial, intraperitoneal and subcutaneous. Preferably the antibody is administered by injection, intravenously or subcutaneously. In one embodiment the antibody antagonist of GM-CSF is administered subcutaneously. In another embodiment the antibody antagonist of GM-CSF is administered intravenously.

In one embodiment the dose for the first and second period is the same. In one embodiment the dose for the first and second period is different. In one embodiment the dose for the first period is higher than the dose for the second period.

In one embodiment, the antibody of the present invention is administered subcutaneously at a fixed dose. In such “fixed dose” treatment the antibody is administered at a certain, fixed, concentration, i.e. without taking into account a patient's body weight.

In one embodiment the antibody antagonist of GM-CSF is administered at a fixed dose of from 20 mg to 200 mg. In another embodiment the antibody antagonist of GM-CSF is administered at a fixed dose of from 20 mg to 180 mg. In another embodiment the antibody antagonist of GM-CSF is administered at a fixed dose of from 20 mg to 150 mg. In another embodiment the antibody antagonist of GM-CSF is administered at a fixed dose of from 20 mg to 100 mg. In another embodiment the antibody antagonist of GM-CSF is administered at a fixed dose of from 20 mg to 50 mg. In another embodiment the antibody antagonist of GM-CSF is administered at a fixed dose of from 100 mg to 180 mg. In another embodiment the antibody antagonist of GM-CSF is administered at a fixed dose of about 135 mg, for example 135 mg. In a further embodiment the antibody antagonist of GM-CSF is administered at a fixed dose of about 180 mg, for example 180 mg. In another embodiment the antibody antagonist of GM-CSF is administered at a fixed dose of about 180 mg, for example 180 mg. In another embodiment the antibody antagonist of GM-CSF is administered at a fixed dose of about 135 mg, for example 135 mg. In another embodiment the antibody antagonist of GM-CSF is administered at a fixed dose of about 90 mg, for example 90 mg. In another embodiment the antibody antagonist of GM-CSF is administered at a fixed dose of about 45 mg, for example 45 mg. In another embodiment the antibody antagonist of GM-CSF is administered at a fixed dose of about 22.5 mg, for example 22.5 mg.

In one embodiment, the patient receives csDMARD treatment in combination with the first and second periods of the antibody treatment which is continued after the second period. In one embodiment the csDMARD is administered to said patient once a week. The patient may receive one or a combination of csDMARDs and may additionally be in conjunction with glucocorticoids or NSAIDS. In one embodiment the antibody antagonist of GM-CSF is administered in combination with a csDMARD. In one embodiment the csDMARD is methotrexate. In one embodiment methotrexate may be administered orally as capsule, tablet or liquid. In another embodiment methotrexate is administered subcutaneously. In another embodiment methotrexate is administered subcutaneously at a fixed dose of from 7.5-25 mg. In another embodiment methotrexate is administered subcutaneously at a fixed dose of from 15-25 mg.

### EXAMPLES

This is a randomized Phase Ha, multicentre, double-blind, placebo-controlled parallel group study to assess the mechanistic evidence to demonstrate that the GM-CSF signalling pathway is active in subjects with RA. The study is to evaluate the proportion of subjects that achieve DAS28(CRP) remission (DAS28<2.6) following 24 weeks of treatment with MOR103 or matching placebo in adult subjects on concomitant methotrexate therapy.

### Treatment Arms and Duration

Screening period up to four weeks, then 52 week combination dosing with rescue for subjects with insufficient response at Week 12 and Week 24, with a 12 week follow-up visit after the last dose.

Five doses (22.5 mg, 45 mg, 90 mg, 135 mg and 180 mg) of MOR103 vs placebo given by subcutaneous injection weekly for first five weeks, then every other week thereafter until Week 50. MOR103/placebo must be administered on the same day each week ±1 day for the first 5 weekly doses. Following this MOR103/placebo must be administered on the same day EOW ±3 days.

Study Design:

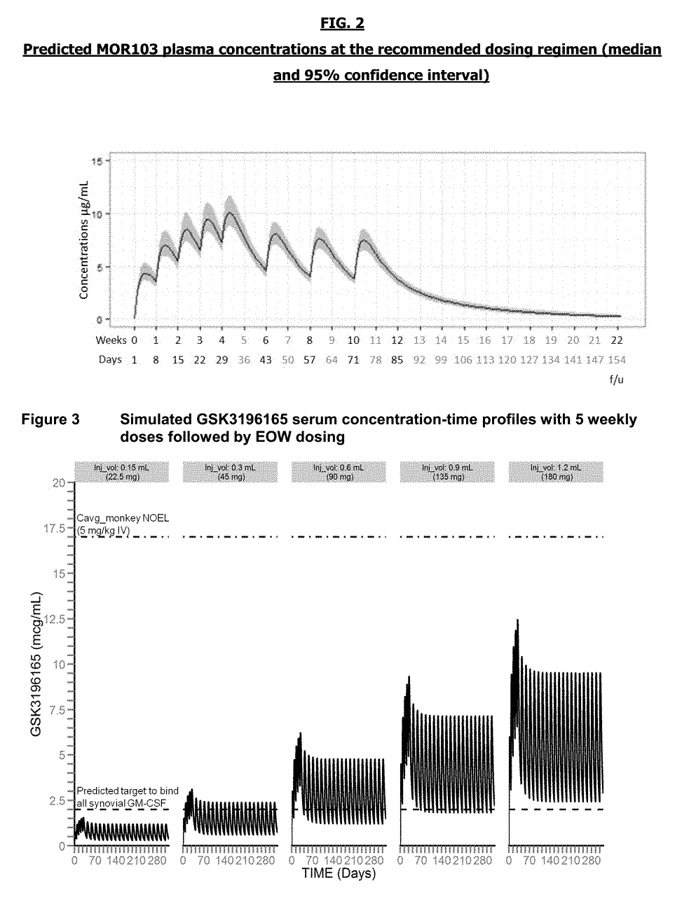

FIG. 3 Demonstrates Simulated MOR103 Serum Concentration-Time Profiles with 5 Weekly Doses Followed by Every Other Week Dosing

Type and Number of Subjects

Approximately 210 subjects with active moderate-severe rheumatoid arthritis despite treatment with methotrexate will be randomized

A placebo arm is included to measure the absolute effect of each dose tested thereby allowing a robust determination of DAS28(CRP) reduction and remission rates, and the dose-response. Inclusion of a placebo arm will also allow a more robust exploration of the safety profile and therapeutic index of MOR103 when given in combination with methotrexate.

All subjects will continue to receive methotrexate, and there are rescue options at specific timepoints built into the study design. In addition, the investigator can withdraw the subject from study at any time as clinically indicated, so subjects having insufficient benefit will not be inadequately treated.

### Inclusion Criteria

### Type of Subject and Diagnosis Including Disease Severity

1. Meets ACR/EULAR 2010 R A Classification Criteria.
2. Functional class I, II or III defined by the 1992 ACR Classification of Functional Status in RA.
3. Disease duration of 12 weeks (time from onset of patient-reported symptoms of either pain or stiffness or swelling in hands, feet or wrists).
4. Swollen joint count of (66-joint count) and tender joint count of (68-joint count) at screening and at Day 1.
5. DAS28(CRP) at screening and DAS28(ESR) at Day 1.
6. C-Reactive Protein (CRP) mg/L at screening.
7. Must have previously received MTX (15-25 mg weekly) for at least 12 weeks before screening, with no change in route of administration, with a stable and tolerated dose for weeks prior to Day 1. A stable dose of MTX mg/week is acceptable, if the MTX dose has been reduced for reasons of documented intolerance to MTX, e.g. hepatic or hematologic toxicity, or per local requirement.

SEQUENCE LISTINGS

SEQ ID NO: 1
GFTFSSYWMN
SEQ ID NO: 2
GIENKYAGGATYYAASVKG
SEQ ID NO: 3
GFGTDF
SEQ ID NO: 4
SGDSIGKKYAY
SEQ ID NO: 5
KKRPS
SEQ ID NO: 6
SAWGDKGM
SEQ ID NO: 7 (variable heavy chain peptide sequence - MOR103)
QVQLVESGGGLVQPGGSLRLSCAASGFTFSSYWMNWVRQAPGKGLEWVSGIENKYAGGATYYAASVKGRFTIS
RDNSKNTLYLQMNSLRAEDTAVYYCARGFGTDFWGQGTLVTVSS
SEQ ID NO: 8 (variable light chain peptide sequence - MOR103)
DIELTQPPSVSVAPGQTARISCSGDSIGKKYAYWYQQKPGQAPVLVIYKKRPSGIPERFSGSNSGNTATLTISGT
QAEDEADYYCSAWGDKGMVFGGGTKLTVLGQ
SEQ ID NO: 9 (variable heavy chain peptide sequence - Mavrilimumab)
QVQLVQSGAEVKKPGASVKVSCKVSGYTLTELSIHWVRQAPGKGLEWMGGFDPEENEIVYAQRFQGRVTMTED
TSTDTAYMELSSLRSEDTAVYYCAIVGSFSPLTLGLWGQGTMVTVSS
SEQ ID NO: 10 (variable light chain peptide sequence - Mavrilimumab)
QSVLTQPPSVSGAPGQRVTISCTGSGSNIGAPYDVSWYQQLPGTAPKLLIYHNNKRPSGVPDRFSGSKSGTSASL
AITGLQAEDEADYYCATVEAGLSGSVFGGGTKLTVL
SEQ ID NO: 11 (heavy chain peptide sequence - Namilumab)
QVQLVQSGAEVKKPGASVKVSCKAFGYPFTDYLLHWVRQAPGQGLEWVGWLNPYSGDTNYAQKFQGRVTMT
RDTSISTAYMELSRLRSDDTAVYYCTRTTLISVYFDYWGQGTMVTVSSASTKGPSVFPLAPSSKSTSGGTAALGC
LVKDYFPEPVTVSWNSGALTSGVHTFPAVLQSSGLYSLSSVVTVPSSSLGTQTYICNVNHKPSNTKVDKKVEPKS
CDKTHTCPPCPAPELLGGPSVFLFPPKPKDTLMISRTPEVTCVVVDVSHEDPEVKFNWYVDGVEVHNAKTKPREE
QYNSTYRVVSVLTVLHQDWLNGKEYKCKVSNKALPAPIEKTISKAKGQPREPQVYTLPPSRDELTKNQVSLTCLV
KGFYPSDIAVEWESNGQPENNYKTTPPVLDSDGSFFLYSKLTVDKSRWQQGNVFSCSVMHEALHNHYTQKSLS
LSPGK
SEQ ID NO: 12 (light chain peptide sequence - Namilumab)
DIQMTQSPSSVSASVGDRVTIACRASQNIRNILNWYQQRPGKAPQLLIYAASNLQSGVPSRFSGSGSGTDFTLTI
NSLQPEDFATYYCQQSYSMPRTFGGGTKLEIKRTVAAPSVFIFPPSDEQLKSGTASVVCLLNNFYPREAKVQWKV
DNALQSGNSQESVTEQDSKDSTYSLSSTLTLSKADYEKHKVYACEVTHQGLSSPVTKSFNRGEC
SEQ ID No 13 Human GM-CSF amino acid sequence (UniProt P04141)
MWLQSLLLLGTVACSISAPARSPSPSTQPWEHVNAIQEARRLLNLSRDTAAEMNET
VEVISEMFDLQEPTCLQTRLELYKQGLRGSLTKLKGPLTMMASHYKQHCPPTPETS
CATQIITFESFKENLKDFLLVIPFDCWEPVQE
SEQ ID No: 14 MOR103_Heavy chain
QVQLVESGGGLVQPGGSLRLSCAASGFTFSSYWMNWVRQAPGKGLEWVSGIENKYAGGAT
YYAASVKGRFTISRDNSKNTLYLQMNSLRAEDTAVYYCARGFGTDFWGQGTLVTVSSAST
KGPSVFPLAPSSKSTSGGTAALGCLVKDYFPEPVTVSWNSGALTSGVHTFPAVLQSSGLY
SLSSVVTVPSSSLGTQTYICNVNHKPSNTKVDKRVEPKSCDKTHTCPPCPAPELLGGPSV
FLFPPKPKDTLMISRTPEVTCVVVDVSHEDPEVKFNWYVDGVEVHNAKTKPREEQYNSTY
RVVSVLTVLHQDWLNGKEYKCKVSNKALPAPIEKTISKAKGQPREPQVYTLPPSREEMTK
NQVSLTCLVKGFYPSDIAVEWESNGQPENNYKTTPPVLDSDGSFFLYSKLTVDKSRWQQG
NVFSCSVMHEALHNHYTQKSLSLSPGK
SEQ ID NO: 15 MOR103_Light chain sequence
DIELTQPPSVSVAPGQTARISCSGDSIGKKYAYWYQQKPGQAPVLVIYKKRPSGIPERFS
GSNSGNTATLTISGTQAEDEADYYCSAWGDKGMVFGGGTKLTVLGQPKAAPSVTLFPPSS
EELQANKATLVCLISDFYPGAVTVAWKADSSPVKAGVETTTPSKQSNNKYAASSYLSLTP
EQWKSHRSYSCQVTHEGSTVEKTVAPTECS
SEQ IN NO: 16; CDRH1 of MOR103 defined by Kabat
SYWMN
SEQ IN NO: 17 CDRH2 of MOR103 defined by Kabat
GIENKYAGGATYYAASVKG
SEQ IN NO: 18 CDRH3 of MOR103 defined by Kabat
GFGTDF
SEQ IN NO: 19: CDRL1 of MOR103 defined by Kabat
SGDSIGKKYAY
SEQ IN NO: 20: CDRL2 of MOR103 defined by Kabat
KKRPS
SEQ IN NO: 21 CDRL3 of MOR103 defined by Kabat
SAWGDKGMV

### BIBLIOGRAPHY

Barrer P. et al., “Synovial macrophage depletion with clodronate-containing liposomes in rheumatoid arthritis”Arthritis Rheum. (2000); 43(9):1951-9.
Chothia C, Lesk A M. Canonical structures for the hypervariable regions of immunoglobulins. J Mol Biol. 1987; 196:901-917.
Cornish A. L. et al.; “G-CSF and GM-CSF as therapeutic targets in Rheumatoid Arthritis”; Nat. Rev. Rheumatol. (2009); 5(10): 554-9.
Emery P. et al.; “Comparison of methotrexate monotherapy with a combination of methotrexate and etanercept in active, early, moderate to severe rheumatoid arthritis (COMET): a randomised, double-blind, parallel treatment trial”; The Lancet (2008) 372(9636): 375-382.
Fleetwood A. J., et al.; “Granulocyte-macrophage colony stimulating factor (CSF) and macrophage CSF-dependent macrophage phenotypes display differences in cytokine profiles and transcription factor activities: implications for CSF blockade in inflammation”; J. Immunol. (2007); 178(8):5245-52.
Gabriel S. E.; “The Epidermiology of Rheumatoid Arthritis”; Rheum. Dis. Clin. North Am. (2001); 27(2): 269-81.
Gasson J. C.; “Molecular physiology of granulocyte-macrophage colony-stimulating factor”; Blood (1991); 77(6): 1131-45.
Hamilton J. A.; “Colony-stimulating factors in inflammation and autoimmunity”; Nat. Rev. Immunol. (2008); 8(7): 533-44.
Hamilton J. A. et al.; “Colony stimulating factors and myeloid cell biology in health and disease” Trends Immunol. (2013); 34(2): 81-9.
Hart P. H. et al.; “Activation of Human Monocytes by Granulocyte-Macrophage Colony-Stimulating Factor: Increased Urokinase-type Plasminogen Activator Activity”; Blood (1991); 77(4): 841-8.
Kabat E A, Wu T T, Bilofsky H, Reid-Miller M, Perry H. Sequence of proteins of immunological interest. Bethesda: National Institute of Health; 1983. 323
Kashiwagi, N. et al.; “Anti-inflammatory effect of granulocyte and monocyte adsorption apheresis in a rabbit model of immune arthritis”; Inflammation (2002); 26(4): 199-205.
Kinne R. W. et al., “Cells of the synovium in rheumatoid arthritis. Macrophages” Arthritis Res. Ther. (2007); 9(6):224.
Martire M. V. et al.; “Factores asociados a remisión sostenida en pacientes con artritis reumatoide” Reumatol. Clin. 2015; 11: 237-241.
Mantovani A. et al.; “New vistas on macrophage differentiation and Activation” Eur. J. Immunol. (2007); 37: 14-6.
Mulherin D. et al.; “Synovial tissue macrophage populations and articular damage in rheumatoid arthritis”; Arthritis Rheum. (1996); 39(1):115-24.
Scheinfeld N.; “A comprehensive review and evaluation of the side effects of the tumor necrosis factor alpha blockers etanercept, infliximab and adalimumab”; J. Dermatolog. Treat. (2004); 15(5): 280-94.
Smolen J. S. et al.; “EULAR recommendations for the management of rheumatoid arthritis with synthetic and biological disease-modifiying antirheumatic drugs: 2013 update”; Ann. Rheum. Dis. (2014); 73; 492-509.
Tak P. P. et al.; “The pathogenesis and prevention of joint damage in rheumatoid arthritis: advances from synovial biopsy and tissue analysis”, Arthritis Rheum. (2000); 43(12): 2619-33.
Tak P. P. et al.; “Is early rheumatoid arthritis the same disease process as late rheumatoid arthritis?”, Best Pract Clin. Rheumatol. (2001); 15(1): 17-26.

1. A method for the treatment of RA in a subject comprising administration to the subject an effective amount of an antibody antagonist of GM-CSF, wherein said antibody is administered to said patient according to the following treatment regimen:
i. a first period wherein the antibody is administered once a week; and
ii. a second period wherein the antibody is administered every other week and then ceased once said patient has sustained remission for a continuous period of at least two months.

2. The method for treatment according to claim 1, wherein remission is maintained after the second period for at least six months while treatment with the antibody is ceased.

3. The method for treatment according to claim 1, wherein remission is maintained after the second period for at least one year while treatment with the antibody is ceased.

4. The method for treatment according to claim 1, wherein the first period is five weeks.

5. The method for treatment according to claim 1, wherein the second period is from one to two years.

6. The method for treatment according to claim 1, wherein RA is early RA.

7. The method for treatment according to claim 1, wherein the patient is csDMARD-naïve before commencing treatment.

8. The method for treatment according to claim 1, wherein the antibody is specific for GM-CSF.

9. The method for treatment according to claim 8, wherein said antibody specific for GM-CSF is an antibody comprising an HCDR1 region of sequence GFTFSSYWMN (SEQ ID NO.: 1), an HCDR2 region of sequence GIENKYAGGATYYAASVKG (SEQ ID NO.: 2), an HCDR3 region of sequence GFGTDF (SEQ ID NO.: 3), an LCDR1 region of sequence SGDSIGKKYAY (SEQ ID NO.: 4), an LCDR2 region of sequence KKRPS (SEQ ID NO.: 5), and an LCDR3 region of sequence SAWGDKGM (SEQ ID NO.: 6).

10. The method for treatment according to claim 8, wherein said antibody specific for GM-CSF is an antibody comprising a heavy chain peptide sequence according to SEQ ID NO: 11 and a light chain peptide sequence according to SEQ ID NO: 12.

11. The method for treatment according to claim 1, wherein the antibody is specific for the GM-CSF receptor.

12. The method for treatment according to claim 11, wherein said antibody specific for the GM-CSF receptor is an antibody comprising a variable heavy chain peptide sequence according to SEQ ID NO: 9 and a variable light chain peptide sequence according to SEQ ID NO: 10.

13. The method for treatment according to claim 1, wherein said antibody is administered at a fixed dose of from 20 mg to 200 mg.

14. The method for treatment according to claim 1, wherein said antibody is administered subcutaneously.

15. The method for treatment according to claim 1, wherein the patient receives csDMARD treatment in combination with the antibody treatment which is continued after the second period.

16. The method for treatment according to claim 1, wherein the csDMARD is administered to said patient once a week.

17. The method for treatment according to claim 16, wherein said csDMARD is methotrexate.

18. The method for treatment according to claim 1, wherein said antibody is administered intravenously.


SEQUENCE LISTING (21 sequences)

SEQ ID NO: 1 - protein, 10 aa, Artificial Sequence
  Amino acid sequence identified using molecular biology techniques.
GFTFSSYWMN

SEQ ID NO: 2 - protein, 19 aa, Artificial Sequence
  Amino acid sequence identified using molecular biology techniques.
GIENKYAGGATYYAASVKG

SEQ ID NO: 3 - protein, 6 aa, Artificial Sequence
  Amino acid sequence identified using molecular biology techniques.
GFGTDF

SEQ ID NO: 4 - protein, 11 aa, Artificial Sequence
  Amino acid sequence identified using molecular biology techniques.
SGDSIGKKYAY

SEQ ID NO: 5 - protein, 5 aa, Artificial Sequence
  Amino acid sequence identified using molecular biology techniques.
KKRPS

SEQ ID NO: 6 - protein, 8 aa, Artificial Sequence
  Amino acid sequence identified using molecular biology techniques.
SAWGDKGM

SEQ ID NO: 7 - protein, 117 aa, Artificial Sequence [sequence omitted]

SEQ ID NO: 8 - protein, 106 aa, Artificial Sequence
  variable light chain peptide sequence - MOR103.
DIELTQPPSVSVAPGQTARISCSGDSIGKKYAYWYQQKPGQAPVLVIYKKRPSGIPERFSGSNSGNTATLTISGTQAEDEADYYCSAWGDKGMVFGGGTKLTVLGQ

SEQ ID NO: 9 - protein, 120 aa, Artificial Sequence
  variable heavy chain peptide sequence - Mavrilimumab.
QVQLVQSGAEVKKPGASVKVSCKVSGYTLTELSIHWVRQAPGKGLEWMGGFDPEENEIVYAQRFQGRVTMTEDTSTDTAYMELSSLRSEDTAVYYCAIVGSFSPLTLGLWGQGTMVTVSS

SEQ ID NO: 10 - protein, 111 aa, Artificial Sequence
  variable light chain peptide sequence - Mavrilimumab.
QSVLTQPPSVSGAPGQRVTISCTGSGSNIGAPYDVSWYQQLPGTAPKLLIYHNNKRPSGVPDRFSGSKSGTSASLAITGLQAEDEADYYCATVEAGLSGSVFGGGTKLTVL

SEQ ID NO: 11 - protein, 449 aa, Artificial Sequence
  heavy chain peptide sequence - Namilumab.
QVQLVQSGAEVKKPGASVKVSCKAFGYPFTDYLLHWVRQAPGQGLEWVGWLNPYSGDTNYAQKFQGRVTMTRDTSISTAYMELSRLRSDDTAVYYCTRTTLISVYFDYWGQGTMVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 12 - protein, 214 aa, Artificial Sequence
  light chain peptide sequence - Namilumab.
DIQMTQSPSSVSASVGDRVTIACRASQNIRNILNWYQQRPGKAPQLLIYAASNLQSGVPSRFSGSGSGTDFTLTINSLQPEDFATYYCQQSYSMPRTFGGGTKLEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 13 - protein, 144 aa, Artificial Sequence
  Human GM-CSF amino acid sequence (UniProt P04141).
MWLQSLLLLGTVACSISAPARSPSPSTQPWEHVNAIQEARRLLNLSRDTAAEMNETVEVISEMFDLQEPTCLQTRLELYKQGLRGSLTKLKGPLTMMASHYKQHCPPTPETSCATQIITFESFKENLKDFLLVIPFDCWEPVQE

SEQ ID NO: 14 - protein, 447 aa, Artificial Sequence
  MOR103 Heavy chain.
QVQLVESGGGLVQPGGSLRLSCAASGFTFSSYWMNWVRQAPGKGLEWVSGIENKYAGGATYYAASVKGRFTISRDNSKNTLYLQMNSLRAEDTAVYYCARGFGTDFWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKRVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 15 - protein, 210 aa, Artificial Sequence
  MOR103_Light chain sequence.
DIELTQPPSVSVAPGQTARISCSGDSIGKKYAYWYQQKPGQAPVLVIYKKRPSGIPERFSGSNSGNTATLTISGTQAEDEADYYCSAWGDKGMVFGGGTKLTVLGQPKAAPSVTLFPPSSEELQANKATLVCLISDFYPGAVTVAWKADSSPVKAGVETTTPSKQSNNKYAASSYLSLTPEQWKSHRSYSCQVTHEGSTVEKTVAPTECS

SEQ ID NO: 16 - protein, 5 aa, Artificial Sequence
  CDRH1 of MOR103 defined by Kabat.
SYWMN

SEQ ID NO: 17 - protein, 19 aa, Artificial Sequence
  CDRH2 of MOR103 defined by Kabat.
GIENKYAGGATYYAASVKG

SEQ ID NO: 18 - protein, 6 aa, Artificial Sequence
  CDRH3 of MOR103 defined by Kabat.
GFGTDF

SEQ ID NO: 19 - protein, 11 aa, Artificial Sequence
  CDRL1 of MOR103 defined by Kabat.
SGDSIGKKYAY

SEQ ID NO: 20 - protein, 5 aa, Artificial Sequence
  CDRL2 of MOR103 defined by Kabat.
KKRPS

SEQ ID NO: 21 - protein, 9 aa, Artificial Sequence
  CDRL3 of MOR103 defined by Kabat.
SAWGDKGMV